(19) United States
(12) Patent Application Publication
(10) Pub. No.: US 2007/0244397 A1
(43) Pub. Date: Oct. 18, 2007

(54) Blood pressure sphygmomanometer integrated into a common apparatus

(75) Inventors: Phillip L. Lam, Monterey Park, CA (US); Ping Cheng Benjamin Liu, San Gabriel, CA (US)

Correspondence Address: LEONARD TACHNER, A PROFESSIONAL LAW;CORPORATION, 17961 SKY PARK CIRCLE, SUITE 38-E, IRVINE, CA, 92614, US

(73) Assignee: CalHealth, Inc.

(21) Appl. No.: 11/799,391

(22) Filed: Apr. 30, 2007

Related U.S. Application Data
(63) Continuation-in-part of application No. 11/418,639, filed on May 5, 2006.
Application No. 11/418,639 is a division of application No. 11/215,983, filed on Aug. 31, 2005, now Pat. No. 7,066,890.
(60) Provisional application No. 60/857,099, filed on Nov. 6, 2006.

(51) Int. Cl. A61B 5/02

(52) U.S. Cl. 600/499

(57) ABSTRACT

A sphygmomanometer cuff assembly, air pump, pressure sensor and release valve are contained in an otherwise conventional apparatus. Alternative embodiments are illustrated and discussed herein. In one such embodiment the sphygmomanometer cuff is nominally positioned within the apparatus structure and is extended outside the apparatus housing during the measurement. In another embodiment, the cuff is always external of the apparatus structure and is easily connected to the apparatus at special ports during the measurement. In yet another embodiment, the cuff is always internal of the apparatus structure and is readily accessible through an aperture in the housing surface of the apparatus to permit the measurement to take place. Preferably, in each of these alternative embodiments a hinged or slidable door or panel protects the cuff or cuff ports between measurements. In all of the embodiments shown herein, the sphygmomanometer cuff is configured for receiving a human finger in circumambient pressured engagement using controlled air pressure to vary the cuff/finger engagement pressure in a precise manner. The apparatus may be any known system that provides a housing that is sufficiently large, a microprocessor for operating on cuff-derived data and a display for presenting blood pressure results in readable form. A typical apparatus for use herein may be for example, a video game controller, a kitchen appliance, an exercise machine, an office machine or a vehicle dashboard.

### CROSS-REFERENCE TO RELATED APPLICATIONS

This application takes priority from U.S. Provisional Patent Application Ser. No. 60/857,099 filed Nov. 6, 2006 which is a continuation-in-part of U.S. patent application Ser. No. 11/418,639 filed on May 5, 2006, which is a divisional of U.S. patent application Ser. No. 11/215,983 filed Aug. 31, 2005 and now issued U.S. Pat. No. 7,066,890.

### BACKGROUND OF THE INVENTION

1. Field of the Invention

The present invention relates generally to blood pressure monitoring devices. More specifically, the invention hereof relates to an otherwise conventional apparatus which incorporates a blood pressure finger cuff for monitoring blood pressure and transmitting measured data to a processor with which the apparatus is associated. Such data is then displayed on the display of the apparatus to provide an easily understood representation of the measured parameters.

2. Background Art

Hypertension is one of modern society's most insidious diseases. Left untreated, it causes life-threatening problems including atherosclerosis, strokes and aneurysms. Long-term hypertension can result in diminished cardio-vascular and kidney function. Yet while high blood pressure is one of the simplest problems to detect in an entirely non-invasive process, it is all too commonly undetected in a large portion of the population. In the past twenty years or so, with the advent of low-cost microprocessor chips and miniature digital electronics and electromagnetic devices, the self-measurement of blood pressure by non-medical personnel has become more readily available. Nevertheless, blood pressure sphygmomanometers are still too sophisticated for a large segment of the population. Even those who are entirely capable of operating such devices tend to put them away in drawers and other out of the way places where they often remain mostly forgotten and unused.

It would be highly advantageous to the early detection of hypertension and thus to the general health of the population if there were a way to provide prolific availability of blood pressure monitoring devices that were readily accessible and even easier to use than currently available digital sphygmomanometers. Having a blood pressure sphygmomanometer at virtually every processor-controlled apparatus with a pressure cuff always immediately adjacent to each such apparatus, would go a long way to serving such an advantageous function. Such is the purpose of the present invention.

### SUMMARY OF THE INVENTION

The present invention combines the blood pressure sphygmomanometer with many commonly available devices of the modern world. Moreover, the invention incorporates the sphygmomanometer cuff into such devices in a manner which makes it extremely simple and convenient to initiate and carry out the blood pressure measurement process. Three alternative embodiments are illustrated and discussed herein. In one such embodiment the sphygmomanometer cuff is nominally positioned within the apparatus structure and is extended outside the apparatus housing during the measurement. In another embodiment, the cuff is always external of the apparatus structure and is easily connected to the apparatus at special ports during the measurement. In yet another embodiment, the cuff is always internal of the apparatus structure and is readily accessible through an aperture in the housing surface of the apparatus to permit the measurement to take place. Preferably, in each of these alternative embodiments a hinged or slidable door or panel protects the cuff or cuff ports between measurements. In all of the embodiments shown herein, the sphygmomanometer cuff is configured for receiving a human finger in circumambient pressured engagement using controlled air pressure to vary the cuff/finger engagement pressure in a precise manner.

In a well-known manner used in blood pressure sphygmomanometers of all kinds, the pressure of the cuff/finger engagement is initially increased until arterial vessel pulsation is beyond cutoff (total occlusion of the artery). The cuff pressure is then slowly decreased until first detection of arterial vessel pulsation (commonly known as Korotkoff Phase I). The corresponding cuff pressure at this point will be substantially equal to systolic blood pressure which is one significant parameter to be monitored. As cuff pressure continues to be decreased, arterial vessel pulsations will eventually become undetectable through the cuff because of the lack of adequate cuff pressure to sense those pulsations (commonly known as Korotkoff Phase V). The pressure of the cuff at this point will be substantially equal to diastolic blood pressure which is another significant parameter to be monitored. Moreover, the frequency of pulsations sensed between the systolic and diastolic pressures is measured and will be substantially equal to the heartbeat rate or pulse rate which is also a parameter of some significance to be monitored. These two cuff pressures and the pulsation frequency are the measured parameter data of the process and corresponding digital data are sent to a processor with which the apparatus is already associated.

Computer software well-known in the art, can then utilize this digital data (typically in binary form) to create a suitable display of the measured parameters on the built-in display of the apparatus. Other functions can also be carried out such as recording the data, graphically plotting data over numerous measurements and communicating the data to others such as by means of the internet for example by e-mail to pre-assigned medical personnel.

The principal advantage of the present invention is therefore clearly the proliferation of a simple and convenient way for the great number of apparatus users to have the benefits of frequent blood pressure monitoring. Moreover, because the blood pressure device can be readily integrated into many known systems, this clearly beneficial health-related device can be enjoyed by many apparatus users and their families with relatively little investment. Additionally, society as a whole will benefit from the likely increase in the early detection of hypertension and the resulting timely treatment thereof and prevention of related diseases. Thus it is believed that the present invention has the potential of leading to a profound change in the number of early detected hypertensive individuals.

### BRIEF DESCRIPTION OF THE DRAWINGS

The various embodiments, features and advances of the present invention will be understood more completely hereinafter as a result of a detailed description thereof in which reference will be made to the following drawings:

FIG. 1 is a view of a generic video game controller having an integrated blood pressure measurement device in accordance with one example of the present invention;

FIG. 2 is an enlarged view of the access panel and contained finger cuff of the video game controller of FIG. 1;

FIGS. 3 through 7 are various views of a first embodiment of the invention in which a sphygmomanometer blood pressure cuff is selectively ejected from within a housing of a known apparatus for blood pressure measurement and showing attendant devices for applying occluding pressure to a human finger and releasing the pressure in a precise manner;

FIG. 8 is a view of a second embodiment of the invention in which a sphygmomanometer blood pressure cuff is selectively affixed to ports at the exterior of a video game controller or other known apparatus for the measurement of blood pressure;

FIG. 9 is a view of a third embodiment of the invention in which a sphygmomanometer blood pressure cuff is fixedly positioned within the housing of a video game controller or other known apparatus and is accessible through at least one aperture in the housing for performing the measurement of blood pressure;

FIG. 10 is a view of a vehicle interior showing an example of how the first embodiment hereof could be used in conjunction with the dashboard of a vehicle;

FIG. 11 is a view of a kitchen appliance providing an integrated blood pressure cuff and blood pressure measurement device;

FIG. 12 is a view of an office machine providing a blood pressure finger cuff and blood pressure measurement device;

FIG. 13 is a view of an exercise machine providing a blood pressure finger cuff and blood pressure measurement device;

FIG. 14 is a block diagram of a blood pressure device/known apparatus interface which may be employed in the present invention; and

FIG. 15 is a graphical representation of the type of measured data derived from an integral blood pressure measurement device hereof for calculation of blood pressure based upon the oscillometric technique.

### DETAILED DESCRIPTION OF PREFERRED EMBODIMENTS

Referring to the accompanying figures, it will be seen that a first embodiment of the present invention is integrated into a video game controller 10 which comprises a housing 12, click buttons 13 and 15 and control devices 14. It will be understood that the precise configuration of the game controller hereof may be varied to virtually any of the many conventional designs. Therefore the shape, location and number of click buttons, the control device and the shape of the housing are all shown herein as illustrative only and should not be deemed limiting of the scope hereof. However, what is a significant improvement over conventional video game controllers now follows.

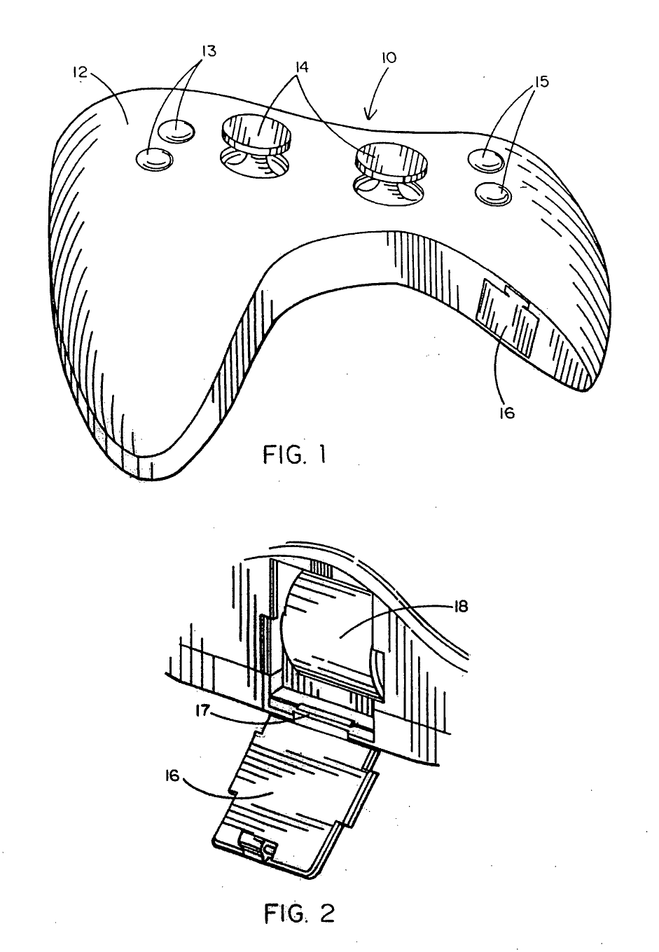

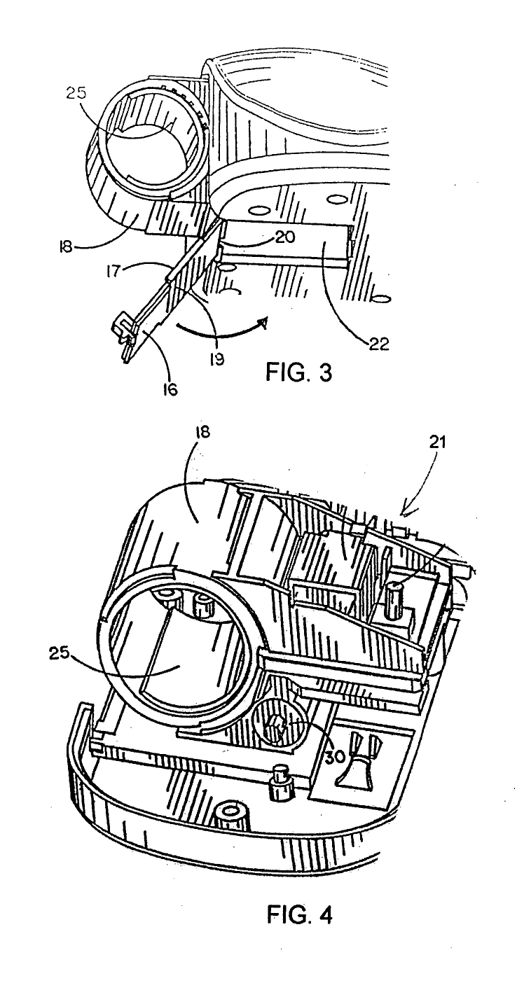

As seen in FIGS. 1 and 2, a hinged door 16 is provided on the side of video game controller housing 12. Hinged door 16, when opened about a hinge 17, leads to the interior of housing 12 where a sphygmomanometer 21 and its cuff assembly 18 are located. As will be hereafter more fully described, upon activation of the sphygmomanometer 21, cuff assembly 18 extends through door 16 to the exterior of the controller housing as shown best in FIG. 3. This extension of the cuff assembly is facilitated by folding door 16 about hinge 17 until it is in alignment with an inner door stage 19 which is, in turn, extended beyond the housing 12 and rotated about its hinge 20 until the door assembly is fully rotated about 270 degrees into a rectangular recess 22 in the bottom surface 24 of the housing as shown in FIG. 3 in particular. When the cuff assembly 18 is fully extended into the position shown in FIG. 3, the cuff assembly is in suitable position for insertion of a person's extended finger for measurement of blood pressure and pulse rate. The assembly may then be re-inserted into the housing and the door assembly rotated back into position for closure of the housing and normal, conventional usage of the video game controller 10.

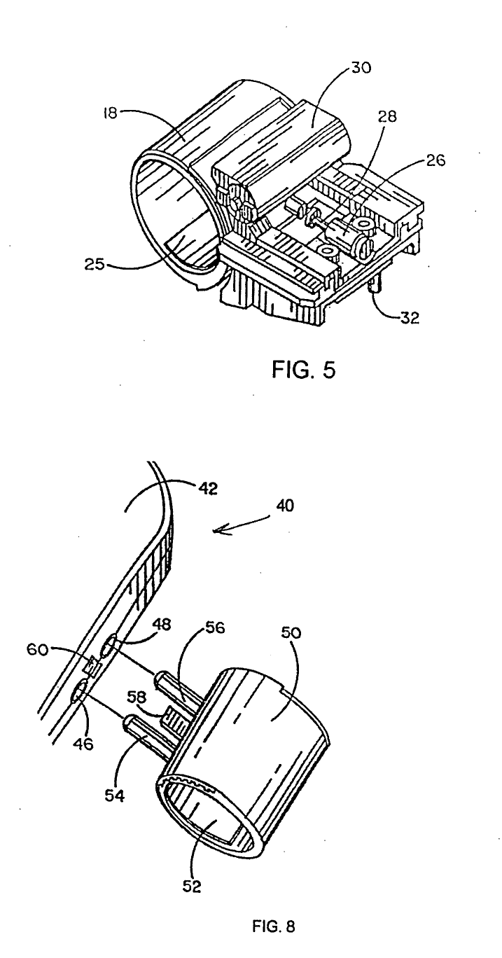

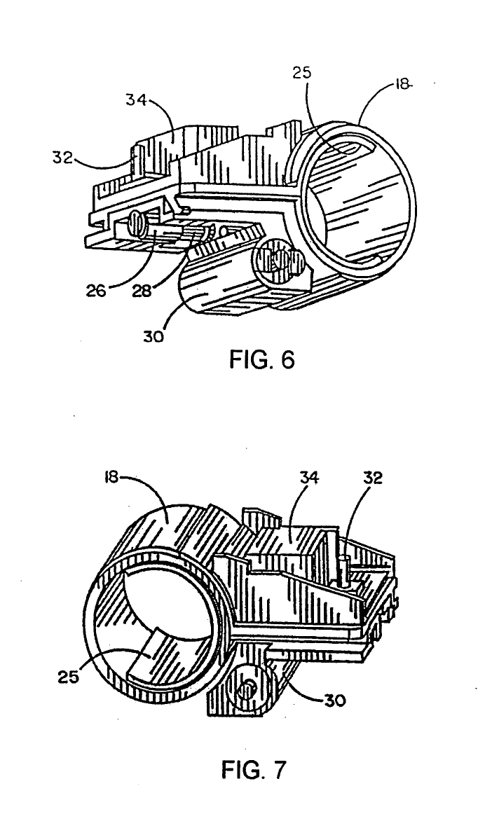

The sphygmomanometer 21 including pressure cuff assembly 18 is shown in FIG. 4 as it sits within the housing 12 and the cuff assembly and attendant components are shown in detail in FIGS. 5 to 7. As seen in those figures, the sphygmomanometer 21 comprises an inflatable air bag 25 within the annular interior of cuff assembly 18. Also forming sphygmomanometer 21 are solenoid 26, extender arm 28, motorized air pump 30, pressure sensor 32 and release valve 34. Solenoid 26 and extender arm 28 provide the ability to extend the cuff assembly 18 out of the housing 12 as shown in FIG. 3. Air pump 30 provides the pressurized air to selectively expand air bag 25 to forcefully grasp a finger protruding through cuff assembly 18. Pressure sensor 32 senses the pressure at the air bag/finger surface interface and release valve 34 releases air from the air bag 25 at a rate that is commensurate with sensor 32 oscillometrically sensing the systolic and diastolic blood pressure at the inserted finger.

FIG. 8 illustrates a second embodiment video game controller 40 having a housing 42. In this embodiment, the housing provides jacks 46 and 48 to which an external cuff assembly 50 having an air bag 52, is attached by connectors 54 and 56. A connector brace 58 mates with a brace receptacle 60 to further support the cuff assembly 50. The remaining components of the sphygmomanometer (less extender arm 28 and solenoid 26 which aren't required in this second embodiment) are interior to housing 42. Once the cuff assembly 50 is attached to the controller 40, the operation of the second embodiment is identical to that of the first embodiment 10. A jack cover may be employed to protect the jacks 46 and 48 when the sphygmomanometer is not in use.

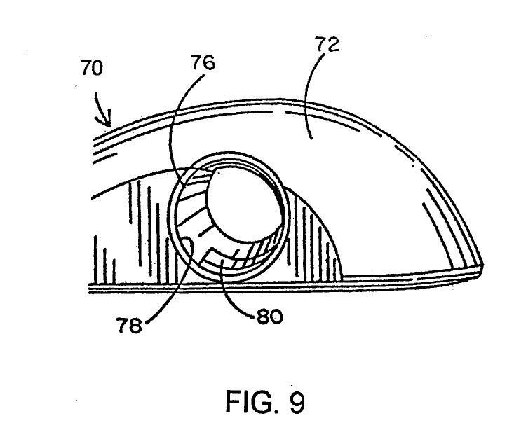

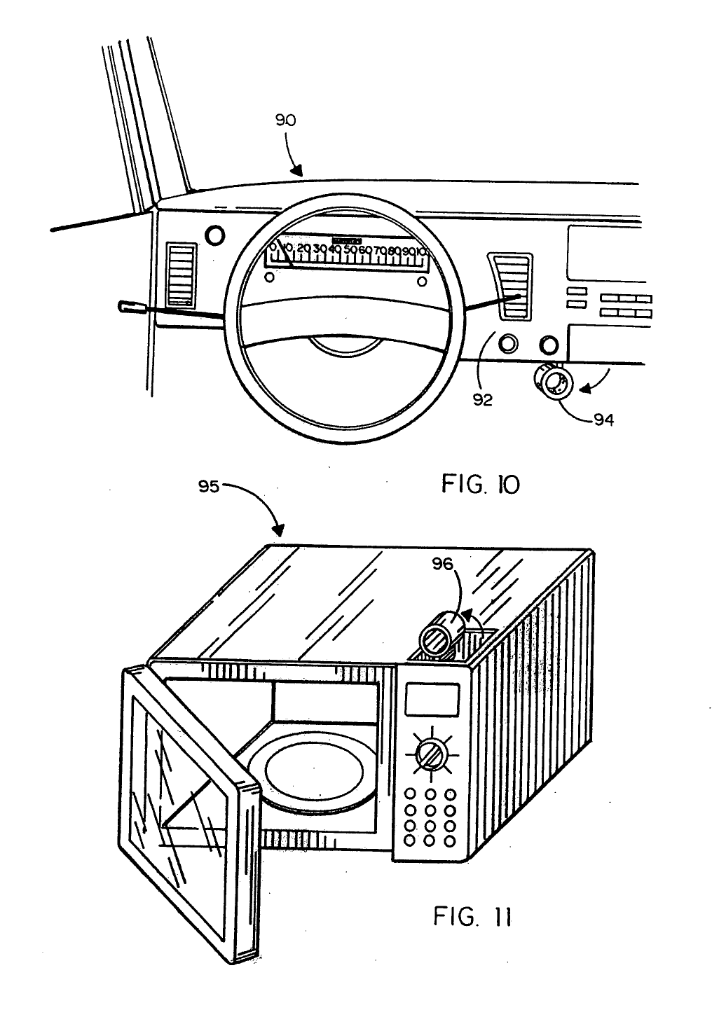

FIG. 9 illustrates a third illustrative embodiment video game controller 70 having a housing 72. In this embodiment a sphygmomanometer is fully contained in a fixed position interior of housing 72 which has apertures 76 axially aligned on opposed side surfaces of the housing. Within apertures 76 is positioned a cuff assembly 78 and an air bag 80 to receive a person's finger for blood pressure and pulse rate measurement as previously described.

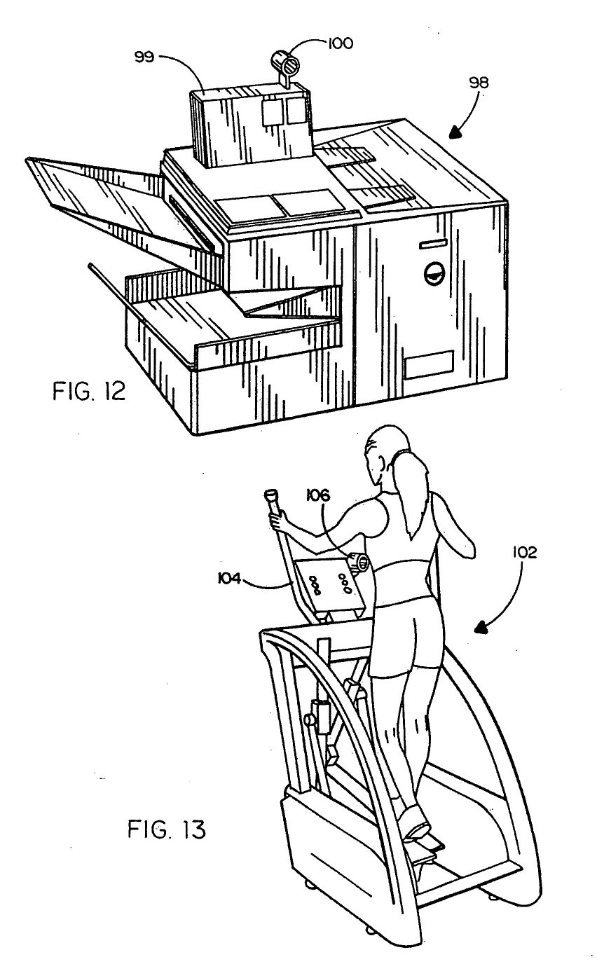

FIGS. 10-13 illustrate other types of otherwise conventional apparatus into which an embodiment of the inventive blood pressure measurement device hereof may be readily integrated to provide a unique new combination. FIG. 10 shows a vehicle interior 90 having a dashboard 92 from which a finger cuff 94 may be extended. As used herein, the term “vehicle” means a car, a truck, an SUV, a bus, a train, an airliner and a ship or boat. FIG. 11 shows a kitchen appliance (here a microwave oven) 95 having a finger cuff 95. FIG. 12 shows an office machine (here a facsimile machine) 98 having a console 99 from which a finger cuff 100 may be extended. FIG. 13 shows an exercise machine 102 having a console 104 from which a finger cuff 106 may be extended for taking blood pressure data from the exerciser.

Each known or conventional apparatus (video game controller, vehicle interior, kitchen appliance, office machine, exercise machine) meet certain criteria for receiving at least one embodiment of the invention for integration therein. Each has a housing console or the like which is sufficiently large to accept the blood pressure measurement device hereof. Each has a microprocessor of sufficient power and speed to derive blood pressure and pulse rate parameters from data provided from use of the attendant pressure cuff. Each has a sufficient display or operates with a display or television to present the parameters in a readily understandable format. Virtually any known apparatus which meets these criteria may be suitable to be combined with a blood pressure measurement embodiment described herein. Therefore, the present invention should be deemed to encompass the combination of a blood pressure measurement device as those described herein, with any known apparatus which has these features. Each such known apparatus may be provided with an activating button or switch to initiate a blood pressure measurement.

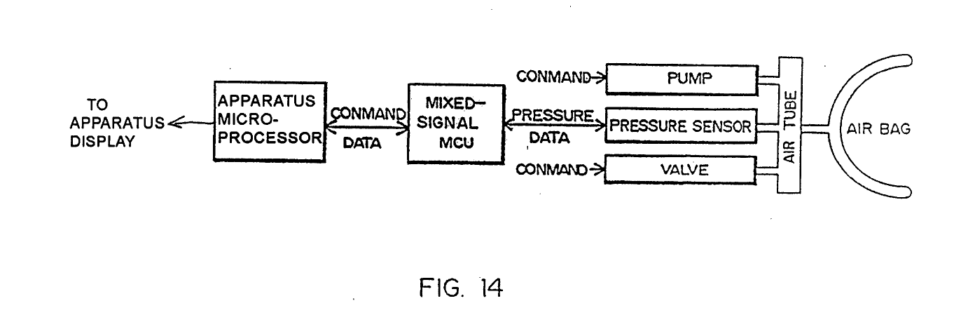

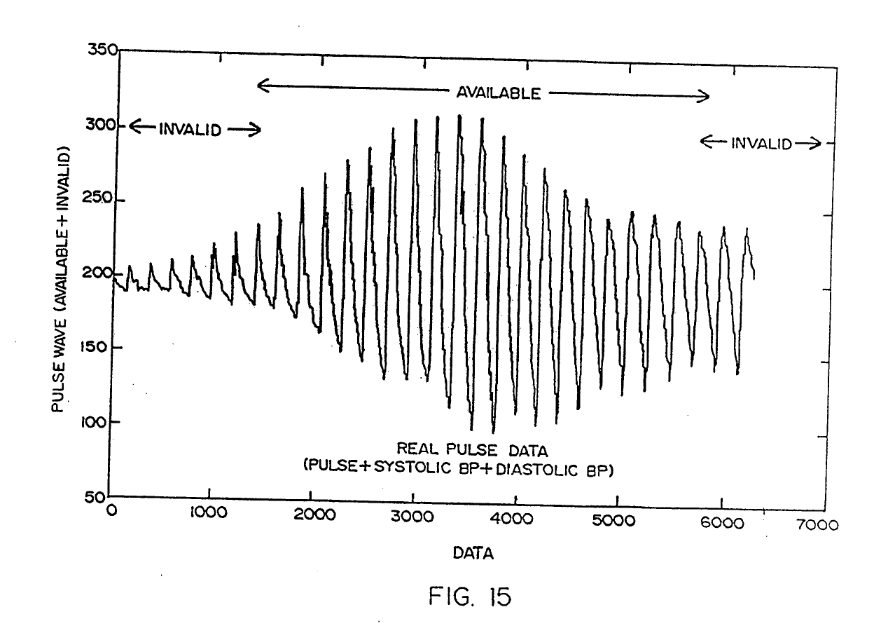

FIG. 14 is a block diagram of the sphygmomanometer apparatus interface showing that the air bag is connected through air tubes to the pump, the pressure sensor and release valve. These components, in turn, receive commands from and provide pressure data to a microprocessor through an MCU. FIG. 15 shows the typical data transmitted to the microprocessor during the blood pressure measurement and from which the systolic, diastolic blood pressure and pulse rate parameters are obtained.

It will now be understood that the present invention provides a novel, convenient way of measuring and tracking health-based parameters by utilizing a modified known apparatus that facilitates obtaining such parameters. While various alternative embodiments have been disclosed herein, other variations will now be perceived by those having the benefit of the description herein. Accordingly, the scope hereof is not limited by the illustrative features described herein, but only by the appended claims and their equivalents.

We claim:

1. The combination comprising:
a known apparatus having a housing, a data processing device and a data display; and
a blood pressure measurement device integrated within said housing and having a blood pressure cuff accessible through an access panel in said housing, said measurement device being connected to said data processing device for deriving blood pressure parameters based on data obtained from said blood pressure cuff, said parameters being displayed on said data display.

2. The combination recited in claim 1 wherein said known apparatus is selected from the group consisting of a video game controller, an electronically-controlled kitchen appliance, an electronically-controlled office machine, an electronically-controlled exercise machine, and a dashboard or console of an electronically-enabled vehicle.

3. A video game controller comprising:
a housing;
a blood pressure measurement device contained within said housing; and
an access door on said housing for gaining access to said blood pressure measurement device for measuring blood pressure of a user of said video game controller.

4. A video game controller comprising:
a housing;
a health parameter measurement device contained within said housing; and
an access door on said housing for gaining access to said measurement device for measuring said health parameter of a user of said video game controller.
